(19) United States
(12) Patent Application Publication
(10) Pub. No.: US 2009/0137948 A1
(43) Pub. Date: May 28, 2009

(54) NEEDLE-FREE INJECTION DEVICE WITH AUTO-DISABLE

(75) Inventors: John R. Marshall, Beaverton, OR (US); Daniel E. Williamson, Sherwood, OR (US); Sergio Landau, Laguna Niguel, CA (US)

Correspondence Address: KOLISCH HARTWELL, P.C., 200 PACIFIC BUILDING, 520 SW YAMHILL STREET, PORTLAND, OR, 97204, US

(73) Assignee: Bioject Inc., Tualatin, OR (US)

(21) Appl. No.: 11/945,212

(22) Filed: Nov. 26, 2007

(51) Int. Cl. A61M 5/30

(52) U.S. Cl. 604/68

(57) ABSTRACT

Needle-free injection devices having a body, an actuation system, and a delivery system including a drive assembly and configured to receive a nozzle assembly. In some embodiments, the drive assembly includes a spring and a support member configured to restrict movement of the spring in a direction non-parallel to a central axis of the spring. In some embodiments, the drive assembly includes a transmission assembly configured to couple a drive source with the actuation system upon engagement of a nozzle assembly with the drive assembly. In some embodiments, the body is configured to acoustically seal an interface between the delivery system and the actuation system. In some embodiments, the body includes a gripping member selectively extendable from the body.

### CROSS-REFERENCE TO RELATED APPLICATIONS

This application relates to U.S. patent application entitled “NEEDLE-FREE INJECTION DEVICE WITH NOZZLE AUTO-DISABLE,” filed Nov. 26, 2007, the disclosure of which is incorporated herein by reference.

### BACKGROUND

Needle-free injection systems provide an alternative to standard fluid delivery systems, which generally use a needle adapted to penetrate the outer surface of a target. Typically, needle-free injection systems are designed to eject the fluid from a fluid chamber with sufficient pressure to allow the fluid to penetrate the target to the desired degree. For example, common applications for needle-free injection systems include delivering intradermal, subcutaneous and intramuscular injections into or through a recipient's skin. For each of these applications, the fluid must be ejected from the system with sufficient pressure to allow the fluid to penetrate the tough exterior dermal layers of the recipient's skin.

One method of generating sufficient pressure is to use a spring powered device, such as those described in U.S. Pat. Nos. 4,592,742, 5,062,830, 5,782,802, and 6,506,177 and U.S. Published Patent Application No. 2005/0119608 A1, the disclosures of which are incorporated herein by reference. Examples of other needle-free injection systems and components are found in U.S. Pat. Nos. 4,596,556, 4,790,824, 4,940,460, 4,941,880, 5,064,413, 5,312,335, 5,312,577, 5,383,851, 5,399,163, 5,503,627, 5,505,697, 5,520,639, 5,746,714, 5,782,802, 5,893,397, 5,993,412, 6,096,002, 6,132,395, 6,216,493, 6,264,629, 6,319,224, 6,383,168, 6,415,631, 6,471,669, 6,572,581, 6,585,685, 6,607,510, 6,641,554, 6,645,170, 6,648,850, 6,623,446, 6,676,630, 6,689,093 6,709,427, 6,716,190, 6,752,780, 6,752,781, 6,783,509, 6,935,384, 6,942,645, 6,979,310, 6,981,961, 7,056,300 and 7,156,823; U.S. Patent Application Publication No. 2006/0189927; and International Publication No. WO 00/72908, the disclosures of which are incorporated herein by reference, in their entirety and for all purposes.

### SUMMARY

The present disclosure is directed to needle-free injection devices having an actuation system configured to initiate an injection and a delivery system including a drive assembly and configured to receive and operably engage a nozzle assembly with the drive assembly. The drive assembly is configured to expel an injectate from the nozzle assembly. The device includes a body configured to house the delivery system and the actuation system.

In some embodiments, the drive assembly includes a spring having a central axis and a support member positioned within the spring and configured to restrict movement of the spring in a direction non-parallel to the central axis of the spring.

In some embodiments, the drive assembly includes a transmission assembly configured to selectively couple a drive source with the actuation system. The transmission assembly may include a locking member configured to couple the drive source with the actuation system upon engagement of a nozzle assembly with the drive assembly.

In some embodiments, at least a portion of the body is configured to acoustically seal an interface between the delivery system and the actuation system.

In some embodiments, the body includes at least one gripping member operatively coupled to the drive assembly and selectively extendable from the body and alterable between a first position, in which the at least one gripping member is spaced away from the body, and a second position, in which the at least one gripping member is substantially flush with the body.

The advantages of the disclosed needle-free injection device may be understood more readily after a consideration of the drawings and the Detailed Description.

### BRIEF DESCRIPTION OF THE DRAWINGS

FIG. 1 is a cross-sectional view of an example of a spring-powered needle-free injection device having a delivery system and an actuation system, the device is shown in a neutral configuration.

FIG. 2 is a cross-sectional view of the device of FIG. 1 in a wound configuration in which the spring is compressed.

FIG. 3 is a cross-sectional view of the device of FIG. 1 in an armed configuration in which a nozzle assembly is coupled to the device.

FIG. 4 is a cross-sectional view of the device of FIG. 1 in a dosed configuration in which the device is prepared to deliver an injection.

FIG. 5 is a cross-sectional view of delivery and actuation system components suitable for use with the device of FIG. 1.

FIG. 6 illustrates a preparation assembly suitable for use with the actuation system of FIGS. 1-5.

### DETAILED DESCRIPTION

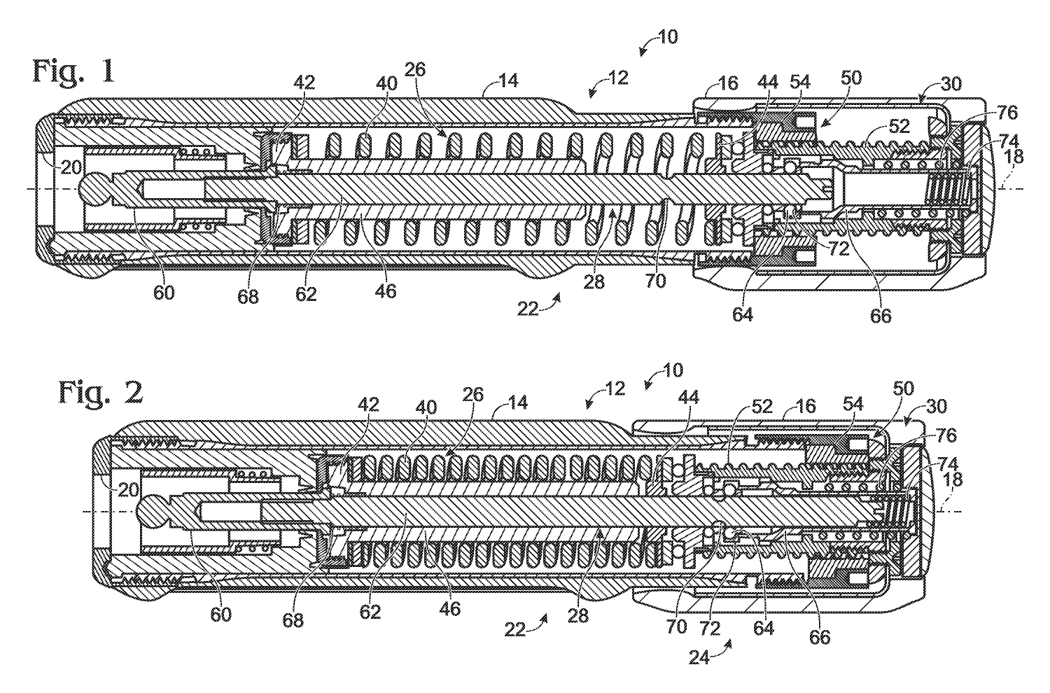

FIGS. 1-4 illustrate an example of a needle-free injection device 10 configured to receive a nozzle assembly 100. Although the disclosed injection device is intended to be reusable, the nozzle assembly may include various auto-disable features to restrict reuse of the nozzle assembly, such as those disclosed in related U.S. patent application entitled “NEEDLE-FREE INJECTION DEVICE WITH NOZZLE AUTO-DISABLE,” filed Nov. 26, 2007. The nozzle may be replaced, for example, after every injection or after a set number of injections.

Device 10 includes a body 12 to enclose various systems used to effect an injection. The body is typically sized and shaped to be comfortably held in a user's hand and may take any suitable configuration. Body 12 may be formed from injection-molded plastic, though various other materials and fabrication methods may be suitable.

As illustrated in FIG. 1, body 12 may be comprised of various subsections, such as housings 14, 16. The housings may be configured to move relative to one another to actuate the various systems. In the example shown in FIGS. 1-4, one or more of the housings may be rotatable relative to another housing and/or rotatable about a central axis 18 to actuate various assemblies of the device.

The body includes an opening 20 in an end of the device to receive the nozzle assembly. The body may include other apertures, such as one or more view ports, to provide feedback or instructions to a user of the device. The apertures may align with indicia, such as arrows or text, that instruct a user in proper operation of the device or convey information to a user, such as the current configuration or status of the device.

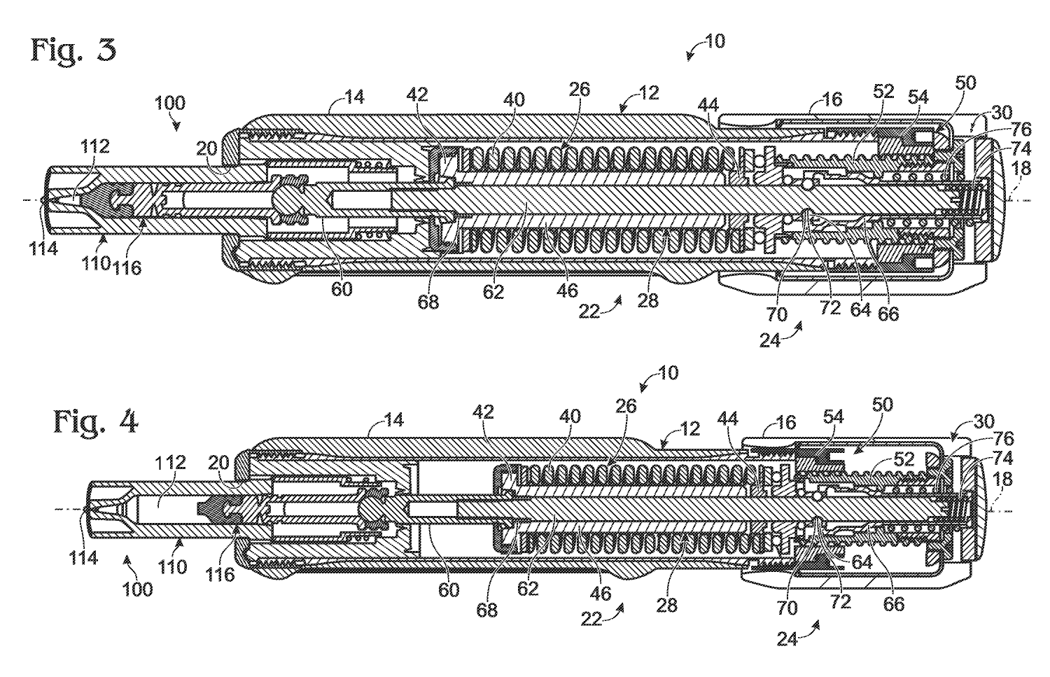

Nozzle assembly 100 is configured to be selectively coupled to the delivery system. The nozzle assembly houses an injectate and provides an interface with a recipient's skin. As illustrated in FIGS. 3 and 4, nozzle assembly 100 includes a nozzle body 110 forming an injectate chamber 112 with one or more outlet orifices 114. The nozzle assembly further includes a plunger 116 configured to move through the injectate chamber toward the orifice(s) to expel an injectate. The plunger may be at least partially visible through the nozzle body. Injectate chamber 112 may include a dose scale (not shown) to incrementally measure the volume of the injectate drawn into the chamber. The dose scale may include indicia or be a pre-molded dose scale having ribs to indicate each unit of measure.

Device 10 may include one or more systems to effect an injection. For example, the device of FIGS. 1-4 includes a delivery system 22 and an actuation system 24. Delivery system 22 provides an interface for delivery of an injectate to a recipient and delivers an injection by expelling the injectate from the device. Delivery system 22 is configured to expel a volume of fluid from the device, such as a drug. The word “drug” as used herein is intended to encompass, for example, and without limitation, any medication, pharmaceutical, therapeutic, vaccine, aesthetic or other material which can be administered by injection. Actuation system 24 prepares the device for delivery of an injection and actuates delivery of an injection.

Delivery system 22 includes a drive assembly 26 to provide a driving force to effect an injection. In some versions of the device, a transmission assembly 28 may be provided to couple the nozzle assembly and the drive assembly.

Actuation system 24 includes a preparation assembly 30 to selectively arrange the drive assembly to provide a drive force to deliver an injection. A trigger assembly 32 assists a user in selectively actuating the drive assembly, directly or indirectly via the transmission assembly, to deliver an injection.

In the illustrative device shown in FIG. 1, drive assembly 26 includes a drive source 40, such as a spring, disposed between spring stop members 42, 44 such that bringing the spring stop members closer together compresses the spring, while decompression of the spring pushes the stop members away from one another. Preparation assembly 30 is actuated by relative rotation between housing sections, such as rotation of housing 16 relative to housing 14. The preparation assembly urges the distal spring stop 44 (i.e., the spring stop furthest from the outlet orifice(s)) towards the proximal spring stop 42 (i.e., the spring stop closest to the outlet orifice(s)) to compress the spring. When the spring is compressed, the device is referred to as being in a wound configuration. As shown, the spring may be aligned with central axis 18. The drive assembly may include a support member 46 positioned through the coils of the spring along the central axis and configured to restrict movement of the spring in a direction non-parallel to the central axis. For example, the support member may prevent buckling of the spring when it is compressed, thus preventing damage to the interior components of the device and directing all available force to the plunger. In some versions of the device, the support member and the body 12, such as an interior surface of housing 14, bound and define a travel path to restrict nonlinear movement of spring 40.

Spring 40 may have a diameter larger than that traditionally used for needle-free injection devices. Using a larger diameter spring decreases the spring rate, such as from eighty pounds-force to one hundred pounds-force. A lower spring rate provides a more constant force and therefore a more constant pressure delivery during injection. The resulting injection pressure makes intramuscular injections possible with a spring-powered needle-free injection device.

In the example of FIGS. 1-4, preparation assembly 30 includes a winder 50. The winder may be rotated in a first direction to alter the device to the wound configuration (as shown in FIG. 2) to compress spring 40. The winder may be rotated in a second direction to alter the device to a dosed configuration (as shown in FIG. 4) to retract the plunger. In the illustrative device, the winder translates a screw 52 (secured to housing 16) relative to a nut 54 (secured to housing 14), thereby moving the distal spring stop member. The screw urges the distal spring stop member towards the proximal spring stop member (to the left as shown in FIG. 2) to compress injection spring 40.

The pressure profile may also be altered by providing an auxiliary spring (not shown). Including a second injection spring provides a secondary source of energy. The auxiliary spring may be serially operated with a compression release mechanism separate from that of spring 40. For example, an auxiliary spring may be actuated at or near the end of an injection to increase the injection force and therefore increased the fluid pressure of the injectate. The transition pressure may thereby be enhanced, such as to provide a longer injection time needed to deliver a larger dose of injectate. The auxiliary spring may be compressed at the beginning of an injection sequence, such as during winding of spring 40, to even out the torque load. The auxiliary spring may be allowed to decompress at the end of the injection to shear off a portion of plunger 116, such as part of the auto-disable features of the nozzle assembly described in U.S. patent application entitled “NEEDLE-FREE INJECTION DEVICE WITH NOZZLE AUTO-DISABLE,” filed Nov. 26, 2007. Decompression of the auxiliary spring may be triggered at the end of travel of the primary spring.

As shown in FIG. 3, nozzle assembly 100 may be coupled to the device by placing the nozzle assembly through opening 20 in the device, such as by inserting the nozzle assembly along axis 18. The nozzle body may include one or more guides (not shown) to assist a user in locating the nozzle assembly relative to the device. The guides and opening may be similarly shaped to assist a user in aligning the nozzle assembly. For example, the nozzle body may be configured to be inserted into the device and then rotated to lock the guides into the device.

In the example shown in FIG. 3, insertion of a nozzle assembly alters the configuration of the device so that an injection may be performed. Consequently, the device is disabled (i.e., prevented from releasing spring 40) until a nozzle assembly is engaged. For example, the transmission assembly of FIG. 1 includes a structure that extends along the central axis of the device. This structure may be a unitary structure or may include more than one component. In the example device of FIGS. 1-4, the transmission assembly includes a ram 60 and an elongate member 62, such as a bolt. The nozzle assembly of FIG. 1 moves the transmission assembly 28 to the right which allows one or more locking members 64 to couple the actuation system to the delivery system. For example, the locking member(s) may couple the ram to a bushing 66, which is secured to the trigger assembly and screw. The bushing may be configured to define a path of travel of the bolt. The ram and bolt may be biased to the left by a spring 68. Since movement of the ram is coupled to movement of the proximal spring stop, the spring stop members are then coupled to one another. The plunger and spring stop members may therefore be moved as a single unit. For example, the plunger and spring stop members may be retracted relative to housing 14 to withdraw the ram and plunger, thereby drawing a dose into the nozzle body. The illustrative device of FIGS. 1-4 uses spherical locking member(s) to couple the actuation and delivery systems. In some versions of the device, the locking members may take the form of one or more balls configured to move between a groove in the elongate member and a corresponding groove in the actuation system. As shown in FIGS. 1-5, the bolt includes groove 70 configured to receive a portion of the locking member(s) to couple the bolt to the end of the bushing via an opposing groove 72.

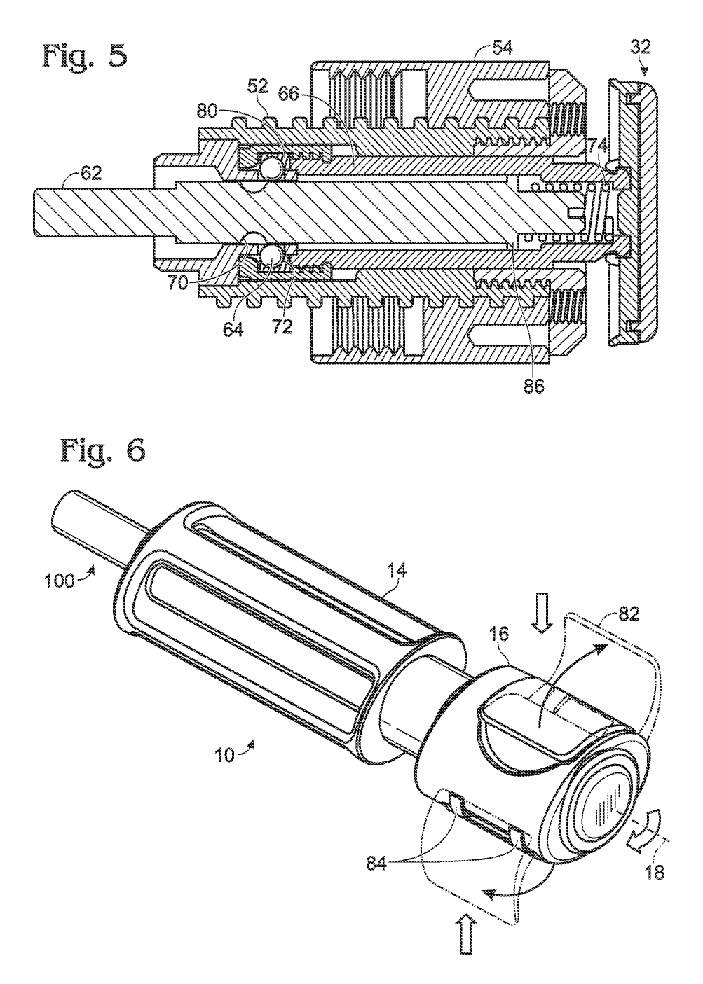

Housing 16 may be rotated in a second direction to withdraw the plunger and both spring stop members. Movement of the plunger to the right, as shown in FIG. 4, draws an injectate into chamber 112 through orifice(s) 114. The device is biased against accidental delivery of an injection by one or more springs. As shown in FIGS. 1-4, the trigger assembly may be biased by a pair of springs 74, 76. As shown in FIG. 5, the trigger assembly may be biased by a single spring 74. In some versions of the device, spring 74 may act as an auxiliary spring configured to provide a secondary source of energy near the end of an injection sequence, as previously discussed.

To deliver an injection, the trigger assembly 32, such as in the form of a button, is actuated to urge the ram and plunger towards the outlet orifice(s). For example, as the trigger assembly in FIG. 1 is moved axially, bushing 66 is urged towards the outlet orifices so that locking members 64 move from groove 70 towards groove 72. The ram is therefore free to travel through the device. Since the distal spring stop member is still fixed relative to body 12, decompression of the spring urges the proximal spring stop member towards the outlet orifice(s). Movement of the proximal spring stop member moves the bolt, ram, and plunger towards the orifice(s) to deliver an injection. At the end of an injection sequence, the device returns to the neutral configuration, as illustrated in FIG. 1.

Actuation of a needle-free injection device without an injectate to act upon may damage the device since the impact of actuation is absorbed by the device components instead of acting on an injectate fluid. This “dry firing” may increase the failure rate of device components, particularly by breaking plastic components. Locking members 64 may assist with disabling the device until a nozzle assembly is properly installed. As shown in FIG. 2, prior to engagement of a nozzle assembly, actuation of trigger assembly 32 does not produce decompression of spring 40. The locking member(s) selectively engage the transmission member to couple the drive assembly to the actuation system in response to insertion of a nozzle assembly.

FIG. 5 illustrates another example of delivery and actuation system components. Prior to coupling of a nozzle assembly with the device, the bolt is biased to the left, as shown in FIG. 5, to preclude the locking members from reaching the groove 70 in the locking bolt. As a nozzle assembly is inserted axially into the device, the plunger engages an end of the ram (shown as a spherical section in FIGS. 1-4). Coupling of a nozzle assembly with the device urges the ram and bolt away from the nozzle assembly, such as to the right with respect to FIG. 5. The grooves 70, 72 are then aligned to receive the locking members 64. In the example shown in FIG. 5, prior to insertion of a nozzle assembly into the device, the bolt is biased to the left, such as by a spring as demonstrated by spring 68 shown in FIGS. 1-4. The locking members are precluded from reaching groove 70 in the bolt until a nozzle assembly is inserted into the device. The length of the bolt may be selected so that the rightward end of the bolt assists in retaining the locking members in groove 72, as best shown in FIG. 1, such as when the device is in the neutral configuration. As shown in FIG. 5, the bolt may include a flange 86 to restrict disassembly of the device, such as by restricting removal of the bolt from the bushing.

Movement of the locking members into groove 70 may produce a loud sound, particularly when both components are formed from metal. One or more shock absorbers 80, as shown in FIG. 5, may be positioned to dampen sounds associated with movement of the locking members. For example, a resilient material such as a urethane ring may be positioned within groove 72 to dampen the sound of the locking members entering groove 72 upon actuation of the device. Additionally or alternatively, bushing 66 may be formed from plastic to restrict noise resonance, also known as “ringing,” generated by movement of the locking members out of engagement with groove 70.

Resilient materials may be used to dampen sound during delivery of an injection in positions other than associated with the locking members. For example, a resilient material may be associated with the trigger assembly. As shown in FIG. 6, an outer covering, such as housing 14, 16 or a coating applied to housings 14, 16, may be formed over at least portions of the device to acoustically seal openings in the device body to reduce sounds emitted during delivery of an injection. This outer covering may be formed from a thermoplastic rubber, such as Santoprene®. In some versions of the device, the body of the device and/or outer covering may be configured to at least partially surround an edge of the trigger assembly that forms an interface between the delivery system and the actuation system.

As previously discussed, the device may be prepared by rotating a portion of the device body, such as by winding housing 16 closer to housing 14. The device may include structure to assist a user in preparing the device. In the example shown in FIG. 6, the preparation assembly includes one or more gripping members 82 that are selectively extendable from the body to assist a user in preparing the device to deliver an injection. The gripping members may be alterable between a first position, in which the gripping members are spaced away from the body, and a second position, in which the gripping members are flush with the body. Altering the gripping members between the first and second positions may assist a user by altering the torque that may be applied to the device during winding. For example, a user with low grip strength, such as from arthritis, may extend the gripping members to increase the length of the moment arm at which a force is applied to wind the device. As shown in FIG. 6, the gripping members may be configured to rotate relative to the body, such as by being hinged to housing 16 at pivots 84. In other configurations, the gripping members may slide, or otherwise selectively extend and/or retract relative to the body.

As shown in FIG. 6, housing 16 is substantially cylindrical. A pair of gripping members 82 extend from opposing sides of the housing so that a user may press against the grips, such as with the thumb and forefinger, to rotate the housing. Although not shown, the gripping members may be configured to rotate about an axis substantially perpendicular to the central axis of the device. For example, the gripping members may be configured to rotate to allow a user to press against the gripping members to wind housing 16 in the opposite direction, such as during dosing.

Although the present invention has been shown and described with reference to the foregoing operational principles and preferred embodiments, it will be apparent to those skilled in the art that various changes in form and detail can be made without departing from the spirit and scope of the invention. The present invention is intended to embrace all such alternatives, modifications and variances. The subject matter of the present invention includes all novel and non-obvious combinations and subcombinations of the various elements, features, functions and/or properties disclosed herein. Inventions embodied in various combinations and subcombinations of features, functions, elements, and/or properties may be claimed through presentation of claims in a subsequent application.

What is claimed is:

1. A needle-free injection device comprising:
an actuation system configured to initiate an injection; and
a delivery system including a drive assembly and configured to receive and operably engage a nozzle assembly with the drive assembly, the drive assembly configured to expel an injectate from the nozzle assembly and including
a drive source; and
a transmission assembly configured to selectively couple the drive source with the actuation system, wherein the transmission assembly includes a locking member configured to couple the drive source with the actuation system upon engagement of a nozzle assembly with the drive assembly.

2. The needle-free injection device of claim 1, wherein the transmission assembly includes an elongate member having a groove configured to receive the locking member, the elongate member being biased to restrict engagement of the groove with the locking member when a nozzle assembly is not engaged with the drive assembly.

3. The needle-free injection device of claim 2, wherein the actuation system includes a shock absorber configured to cushion movement of the locking member out of the groove.

4. The needle-free injection device of claim 2, wherein the locking member includes a ball configured to move between the groove in the elongate member and a corresponding groove in the actuation system.

5. The needle-free injection device of claim 2, wherein the actuation system includes a bushing configured to define a path of travel of the elongate member.

6. The needle-free injection device of claim 1, wherein the transmission assembly includes a ram configured to couple a plunger of a nozzle assembly with the elongate member and thereby transmit a force from the drive source to the plunger.

7. The needle-free injection device of claim 1, wherein the drive source is a spring and the locking member is configured to selectively couple the spring with the actuation system.

8. A needle-free injection device comprising:
an actuation system configured to initiate an injection; and
a delivery system including a drive assembly and configured to operably engage a nozzle assembly with the drive assembly, the drive assembly including
a spring having a central axis and configured to apply a force to deliver an injection; and
a support member positioned within the spring and configured to restrict movement of the spring in a direction non-parallel to the central axis.

9. The needle-free injection device of claim 8, wherein the support member is concentrically positioned within the spring.

10. The needle-free injection device of claim 8, wherein the device includes a body configured to house the delivery system and the actuation system, and the support member and an interior surface of the body bound and define a travel path that restricts nonlinear movement of the spring.

11. The needle-free injection device of claim 8, wherein the drive assembly includes an auxiliary spring configured to provide a secondary force at an end of an injection sequence.

12. A needle-free injection device comprising:
a delivery system configured to deliver an injection by expelling a fluid;
an actuation system configured to initiate delivery of the injection; and
a body configured to house the delivery system and the actuation system, at least a portion of the body configured to acoustically seal an interface between the delivery system and the actuation system.

13. The needle-free injection device of claim 12, wherein the at least a portion of the body is formed from a thermoplastic rubber.

14. The needle-free injection device of claim 12, wherein the actuation system includes a trigger assembly configured to selectively actuate the delivery system and the body is configured to at least partially surround an edge of the trigger assembly that forms the interface.

15. A needle-free injection device comprising:
a delivery system including a drive assembly configured to deliver an injection by expelling a fluid;
an actuation system configured to initiate delivery of an injection; and
a body configured to house the delivery system and the actuation system, the body including at least one gripping member operatively coupled to the drive assembly and selectively extendable from the body and alterable between a first position, in which the at least one gripping member is spaced away from the body, and a second position, in which the at least one gripping member is substantially flush with the body.

16. The needle-free injection device of claim 15, wherein altering the at least one gripping member between the first and second positions alters the length of a moment arm configured to apply torque to the drive assembly.

17. The needle-free injection device of claim 16, wherein the at least one gripping member is configured to pivot relative to the body.

18. The needle-free injection device of claim 15, wherein the at least one gripping member includes a pair of gripping members configured to extend from opposing sides of the body.
